(19) United States
(12) Patent Application Publication
(10) Pub. No.: US 2025/0262203 A1
(43) Pub. Date: Aug. 21, 2025

(54) METHOD FOR TREATING CALCIFIC HEART VALVE DISEASE(CHVD)

(71) Applicant: UNION HOSPITAL TONGJI MEDICAL COLLEGE HUST, Wuhan (CN)

(72) Inventors: Fei LI, Wuhan (CN); Li XU, Wuhan (CN); Yidan ZHENG, Wuhan (CN); Xingyu QIAN; Yuqi LIU, Wuhan (CN); Nianguo DONG, Wuhan (CN)

(21) Appl. No.: 18/889,293

(22) Filed: Sep. 18, 2024

(30) Foreign Application Priority Data
Feb. 18, 2024 (CN) 202410181131.8

(51) Int. Cl. A61K 31/496

(52) U.S. Cl. CPC A61K 31/496

(57) ABSTRACT

A method for treating calcific heart valve disease (CHVD) is provided, belonging to the technical field of medicine. Studies have shown that manidipine intervention in drug screening of mouse osteoclasts can effectively restore an osteoclast function and then dissolve calcium salt deposits, and is expected to reverse valve calcification, thereby fundamentally utilizing economical and practical drugs to treat the CHVD. For the first time, manidipine, a common drug with desirable safety, is applied to the field of valvular calcification. This approach may also have a positive effect on other cardiovascular diseases. The method provides a novel approach for drug treatment of the CHVD.

### CROSS REFERENCE TO RELATED APPLICATION

This patent application claims the benefit and priority of Chinese Patent Application No. 2024101811318, filed with the China National Intellectual Property Administration on Feb. 18, 2024, the disclosure of which is incorporated by reference herein in its entirety as part of the present application.

### TECHNICAL FIELD

The present disclosure belongs to the technical field of medicine, and in particular relates to a method for treating calcific heart valve disease (CHVD) using a product containing manidipine.

### BACKGROUND

Heart valve disease (HVD) is a major disease that affects human health, with approximately 209 million patients worldwide and more than 25 million patients in China. Calcific heart valve disease (CHVD) is the most common type of HVD, with an incidence rate increasing significantly with the aging of population. In developed countries, the incidence rate is 2% to 9% among people over 65 years old and as high as 13% among people aged 75 to 85 years old. The lack of targeted drug control for CHVD is a long-standing unresolved problem in the cardiovascular field, and valve replacement is currently the main treatment method. Existing types of artificial heart valve replacements mainly include mechanical valves and biological valves. The mechanical valve requires lifelong anticoagulation and is prone to cause serious complications such as bleeding and embolism. The biological valve replacement also faces the international problem of calcification and decay, requiring repeated surgeries. Therefore, targeted development of drugs to prevent or delay valvular calcification is of great significance for the control of CHVD. However, there is no effective drug treatment option so far.

Osteoclasts play an important role in calcium deposition, bone formation, and bone remodeling. These cells aggregate near the core of calcium salt deposition, dissolve calcium salts by releasing substances such as cathepsin K, and absorb the salts through lysosome phagocytosis. However, the osteoclasts in human cardiovascular tissues are mostly missing or inactivated, and cannot effectively dissolve abnormal calcification deposits in the cardiovascular system. Therefore, it is of great significance to analyze why the formation of osteoclasts in valve tissue is reduced and their function is inactivated, thus reversing their function in order to improve the valve calcification.

### SUMMARY

A first objective of the present disclosure is to provide a product for treating CHVD, including manidipine.

Preferably, the product is a drug.

A second objective of the present disclosure is to provide use of manidipine in preparation of a product for treating CHVD.

Preferably, the product is a drug.

More preferably, the drug uses the manidipine as a sole active ingredient.

More preferably, the drug further includes a medically acceptable auxiliary material.

More preferably, a dosage form of the drug is any one selected from the group consisting of a tablet, a granule, a capsule, a pill, a solution, an emulsion, and a suspension.

Compared with the prior art, the present disclosure has the following beneficial effects:

For the first time in the present disclosure, manidipine, a common drug with desirable safety, is applied to the field of valvular calcification. This approach may also have a positive effect on other cardiovascular diseases. The method provides a novel approach for drug treatment of the CHVD.

### BRIEF DESCRIPTION OF THE DRAWINGS

FIG. 1 shows the pathogenesis of osteoclast functional inactivation in CHVD;

FIG. 2 shows a clustering diagram of the drug screening results in Example 1;

FIG. 3 shows drug categories to which the 34 most significant drugs in Example 1 belong; and

FIG. 4 shows results of RNAseq sequencing of osteoclasts collected after intervention with three drugs in Example 1, and the enrichment of differentially expressed genes, where DRUG1 represents manidipine; DRUG2 represents manidipine 2HCl; and DRUG3 represents nimodipine.

### DETAILED DESCRIPTION OF THE EMBODIMENTS

### Example 1

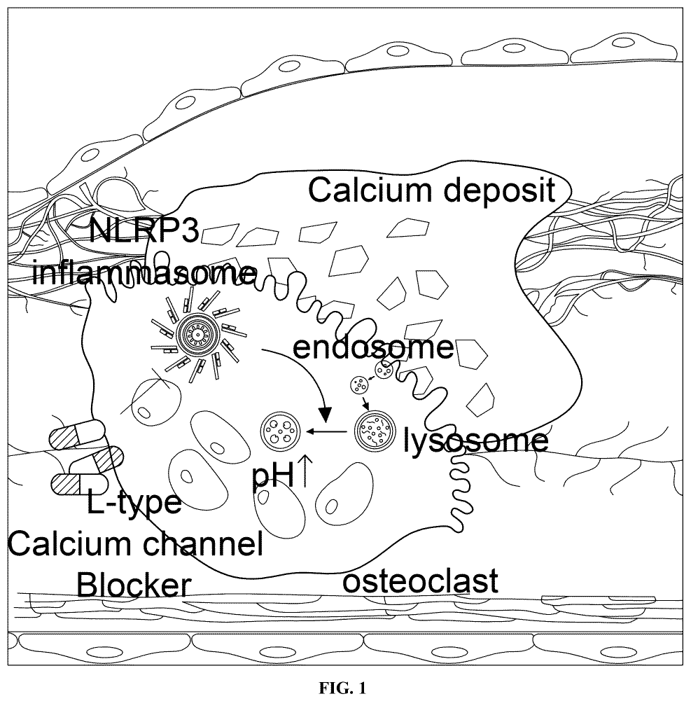

FIG. 1 showed the pathogenesis of osteoclast inactivation in CHVD. The specific pathogenesis was that the activation of NLRP3 inflammasome in osteoclasts in valves affected the activity and endocytosis of their lysosomes, and lost the ability to digest, phagocytose, and degrade the calcium salt deposits formed, thus failing to change the valve calcification.

To explore the reasons for the reduced functional inactivation of osteoclast formation in valve tissue, the proportion and functional status of osteoclasts were carefully explored in valve tissue of CHVD patients. The results showed that NLPRP3 expression was elevated in osteoclasts of CHVD patients, and osteoclast function and formation were reduced. Further cell experiments demonstrated that an important pathogenesis of osteoclast inactivation in CHVD was that increased NLRP3 expression inhibited the expression of cathepsin K and the formation of osteoclasts, thereby hindering the dissolution of existing calcium deposits.

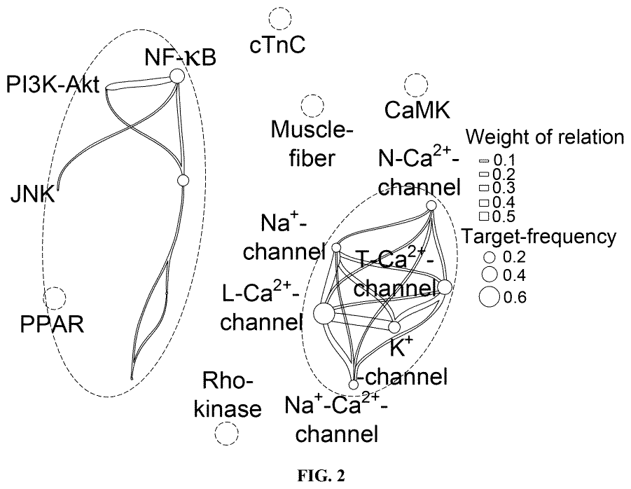

A drug screening experiment was conducted based on the above mechanism. 198 potential NLRP3 inhibitors were applied to osteoclasts, and the expressions of NLRP3 and cathepsin K were detected. After clustering (the results in FIG. 2 showed that among the 198 NLRP3 inflammasome inhibitors, the drugs that could significantly enhance osteoclast function were mainly concentrated in the category of calcium channel blockers, especially in L-type calcium channel blockers) and concentration gradient supplementation experiments, it was finally found that manidipine upregulated tissue proteinase K in mouse osteoclasts while inhibiting NLRP3. This was a novel treatment option that activated osteoclast function to promote its dissolution of calcium salt deposits and thus alleviated or even reversed the CHVD.

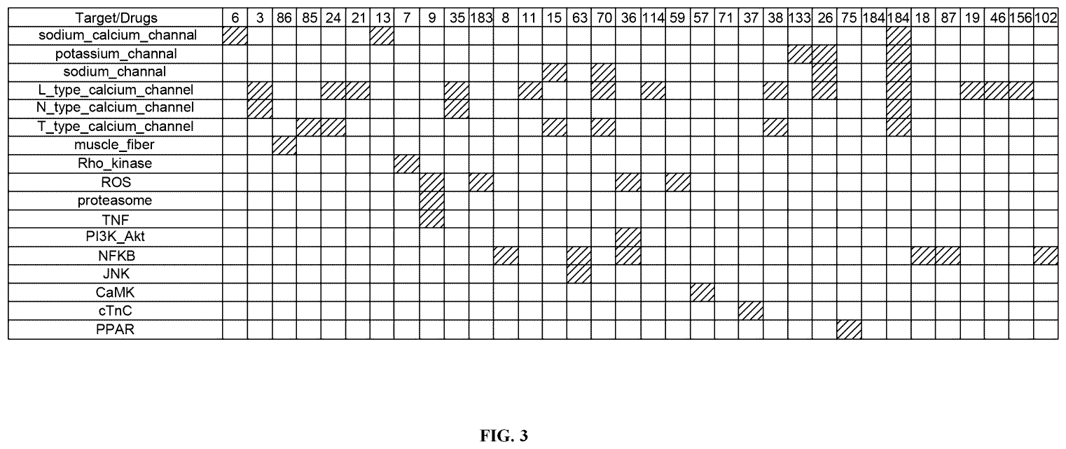

FIG. 3 showed the drug categories to which the most significant 34 drugs belonged. For example, drug 3 was marked in diagonal grid in the category of L-type and N-type calcium channel blockers, indicating that this drug could be classified as L-type calcium channel blockers and N-type calcium channel blockers.

Experimental results had shown that this technical solution could improve inflammatory responses and activate the dissolution of local calcium salt deposits by administering manidipine, which was expected to improve or even reverse the aortic valve calcification and treat the CHVD through simple and economical drugs.

Apolipoprotein e (Apoe)-deficient mice were fed high-fat diet for 24 weeks to establish the recognized hyperlipidemia-induced CHVD model, and 10 of the mice were given NLRP3 gene knockout. The results showed that compared with the control group, the valvular calcification status of the gene knockout intervention group was significantly improved under the background of hyperlipidemia, where there were smaller calcium salt deposition nodules, lower transvalvular flow velocity, and less expression level of calcification-related proteins.

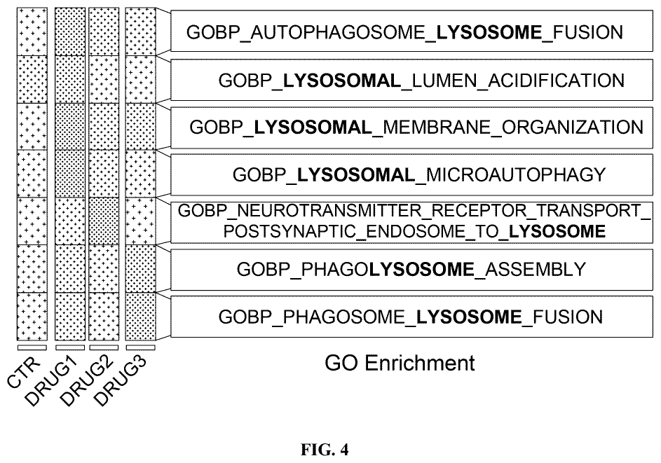

In the cell experiment, drug screening experiments were conducted. The results showed that calcium channel blockers were an important and effective drug category among the interventions of 198 drugs. Further drug screening was conducted on 12 calcium channel blockers (amlodipine, manidipine, manidipine 2HCl, flunarizine 2HCl, cilnidipine, protopine, nimodipine, ranolazine 2HCl, sulfasalazine, amlodipine besylate, isradipine, and nifedipine) with significant effects, so as to determine the optimal drug concentration, of which three with the best effects were selected for RNAseq testing after intervention. A specific process was as follows.
(1) Drug screening: cells were plated in a 6-well plate at a density of 10\hat{ }6 per well. After inducing osteoclast formation for 3 d, 12 drugs (the initial concentration of each drug was 10 mM, dissolved in DMSO) were administered at six concentration gradients of 1:100, 1:500, 1:1000,1:2000, 1:5000, and 1:10000 to allow intervention (1 mL per well) for 3 d. After the intervention, fluorescent antibodies against NLRP3 and cathepsin K were added at a dilution ratio of 1:1000 of the antibody stock solution, followed by high-content fluorescence microscopy detection, and the protein expression level was determined by fluorescence intensity. The three drugs with the lowest NLRP3 fluorescence intensity and the highest cathepsin K fluorescence intensity were selected as the subsequent application drugs.
(2) RNAseq: cells were plated at a density of 10\hat{ }6 per well in a 6-well plate. After inducing osteoclast formation for 3 d, 3 drugs (manidipine, manidipine hydrochloride, and nimodipine, with initial concentration of 10 mM, dissolved in DMSO) were administered for intervention for 3 d (drugs were purchased from the Selleck drug library, diluted in culture medium at 1:1000, and 1 mL was added to each well). After the intervention, the cells in the well plate were washed with PBS, and then 1 mL of Trizol was added to each well of the six-well plate, shaken on a shaker for 10 min, and then a resulting suspension was collected by pipetting into a centrifuge tube and transported on dry ice to BGI to allow transcriptome sequencing. The results were shown in FIG. 4.

The results showed that the osteoclast formation pathway and lysosomal function-related pathways were significantly upregulated after intervention with manidipine. This indicated that manidipine intervention significantly improved the osteoclast formation and dysfunction caused by NLRP3, and was expected to reverse the loss or dysfunction of osteoclasts in calcified valves, thereby improving or even reversing the progression of CHVD.

CHVD is a major and intractable disease that affects human health. However, since the specific biological mechanism of valvular calcification has not been fully elucidated, it is difficult to develop targeted drugs to effectively prevent and treat the CHVD. Cardiovascular risk factors such as hyperlipidemia are believed to be related to valvular calcification, but there has been a lack of in-depth and accurate research on the biological mechanism of how they specifically affect and ultimately lead to valvular calcification. As a frontline clinician and medical researcher, the applicant has revealed and verified new specific mechanisms that may delay or even reverse the onset of CHVD through cell and animal experiments based on clinical problems. For the first time, increased NLRP3 expression in valve tissue inhibits the expression of cathepsin K and the formation of osteoclasts, thereby hindering the dissolution of existing calcium salt deposits. Inhibiting NLRP3 can not only inhibit the inflammatory response that promotes valve calcification, but also increase osteoclast function and promote the dissolution and absorption of existing calcium salt deposits.

Manidipine is a dihydropyridine calcium antagonist. The manidipine has a dose-dependent hypotensive effect in spontaneously hypertensive rats, birenal-clip hypertensive rats, deoxycorticosterone acetate hypertensive rats, and renal hypertensive dogs. The drug has a stronger effect than that of nicardipine and nifedipine and lasts longer. Manidipine is selective for resistance vessels, can dilate renal blood vessels, has less inhibitory effect on the myocardium and conduction, and shows no adverse effect on blood lipids. Studies have shown that manidipine intervention in drug screening of mouse osteoclasts can effectively restore an osteoclast function and then dissolve calcium salt deposits, and is expected to reverse valve calcification, thereby fundamentally utilizing economical and practical drugs to treat the CHVD.

The above examples are only intended to describe the preferred implementations of the present disclosure, but not to limit the scope of the present disclosure. Various alterations and improvements made by those of ordinary skill in the art based on the technical solution of the present disclosure without departing from the design spirit of the present disclosure shall fall within the scope of the appended claims of the present disclosure.

What is claimed is:

1. A method for treating calcific heart valve disease (CHVD), comprising using a product containing manidipine.

2. The method according to claim 1, wherein the product is a drug.

3. The method according to claim 2, wherein the drug uses the manidipine as a sole active ingredient.

4. The method according to claim 3, wherein the drug further comprises a medically acceptable auxiliary material.

5. The method according to claim 3, wherein a dosage form of the drug is any one selected from the group consisting of a tablet, a granule, a capsule, a pill, a solution, an emulsion, and a suspension.

6. The method according to claim 4, wherein a dosage form of the drug is any one selected from the group consisting of a tablet, a granule, a capsule, a pill, a solution, an emulsion, and a suspension.
